(19) United States
(12) Patent Application Publication
(10) Pub. No.: US 2021/0353587 A1
(43) Pub. Date: Nov. 18, 2021

(54) COMPOSITIONS FOR THE TREAMENT OF CANCER AND OTHER CONDITIONS

(71) Applicant: RMD SCIENCES INC., Evanston, IL (US)

(72) Inventors: Akihito TAIRA, Lincolnshire, IL (US); Kazuhiro SHIMOMURA, Wilmette, IL (US)

(21) Appl. No.: 17/289,165

(22) PCT Filed: Nov. 11, 2019
(86) PCT No.: PCT/US2019/060686

§ 371 Date: Apr. 27, 2021

Related U.S. Application Data
(60) Provisional application No. 62/757,913, filed on Nov. 9, 2018.

(51) Int. Cl. A61K 31/351

(52) U.S. Cl. CPC A61K 31/351

(57) ABSTRACT

A composition includes at least one of a modified glucal or galactal molecule. The molecules are useful for the treatment of cancer, particularly in a human patient, more particularly in a human patient exhibiting cell-specific increases in glucose uptake without a corresponding lactate production. The compounds are also useful for treating a human that has hypoxic cells expressing elevated levels of HIT activity, a plurality of cells displaying a lower amplitude of circadian oscillation or an increase in reactive oxygen species in mitochondria of the cells of the human.

### TECHNICAL FIELD

The disclosure is directed to molecules that are useful for the treatment of cancer in a patient, particularly in a human patient, more particularly in a human patient exhibiting cell-specific increases in glucose uptake without a corresponding lactate production, or the human has hypoxic cells expressing elevated levels of HIF activity, or the human possess a plurality of cells displaying a lower amplitude of circadian oscillation or the human has an increase in reactive oxygen species in mitochondria of the cells of the human.

### BACKGROUND

Cancer is a group of varied diseases characterized by uncontrolled growth and spread of abnormal cells. Generally, all types of cancers involve some abnormality in the control of cell growth and division. The pathways regulating cell division and/or cellular communication become altered in cancer cells such that the effects of these regulatory mechanisms in controlling and limiting cell growth fails or is bypassed. Through successive rounds of mutation and natural selection, a group of abnormal cells, generally originating from a single mutant cell, accumulates additional mutations that provide selective growth advantage over other cells, and thus evolves into a cell type that predominates in the cell mass. This process of mutation and natural selection is enhanced by genetic instability displayed by many types of cancer cells, an instability which is gained either from somatic mutations or by inheritance from the germ line. The enhanced mutability of cancerous cells increases the probability of their progression towards the formation of malignant cells. As the cancer cells further evolve, some become locally invasive and then metastasize to colonize tissues other than the cancer cell's tissue of origin. This property along with the heterogeneity of the tumor cell population makes cancer a particularly difficult disease to treat and eradicate.

Worldwide, more than 10 million people are diagnosed with cancer every year and it is estimated that this number will grow to 15 million new cases every year by 2020.

Cancer causes six million deaths every year or 12% of the deaths worldwide. There remains a need for methods that can treat cancer. Herein is provided the basis for pharmaceutical compositions useful in the prevention and treatment of cancer in humans and other mammals.

It has been previously shown that a thermally activated saccharide can be a useful adjuvant for the treatment of cancer in a patient, especially in a human patient. Such treatment and process for creating such a treatment can be found in international patent publication WO/2018/031435, hereby incorporated by reference.

### BRIEF DESCRIPTION OF THE DRAWINGS

FIG. 1 depicts relative transfer efficiencies of known and herein disclosed compounds through a cell membrane and the conversion of the known compounds to those disclosed herein.

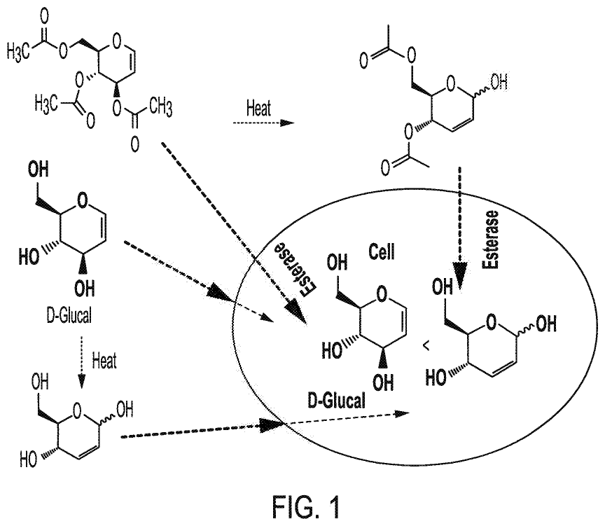

FIG. 2 depicts preferred structures of molecules of the compositions of this disclosure.

FIG. 3 depicts additional methods that can be used to synthesize compositions of this disclosure.

FIG. 4 depicts yet more additional methods that can be used to synthesize compositions of this disclosure.

FIG. 5 depicts dose-response curves of the treatment of normal and cancer cell lines with a composition of this disclosure.

### DETAILED DESCRIPTION

This disclosure is directed to chemotherapeutically active compositions, their manufacture, and use with or without other chemotherapeutic agents. For clarity, the term chemotherapeutically active composition refers to a material that includes a one or more of the compounds as will be described herein. The term chemotherapeutic agent(s) refers to chemical compositions that provide chemotherapeutic effects other than the herein recited compounds.

Herein are described compositions comprising any molecule represented by the following formulas:

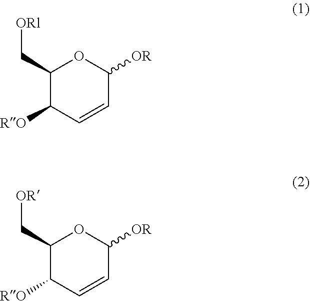

where R, R′, and R″ are independently selected from the group consisting of hydrogen, an organic functionality consisting of carbon with hydrogen, nitrogen and/or oxygen and including from 1 to about 24 carbon atoms, a hetero-organic functionality comprising carbon with hydrogen, nitrogen and/or oxygen, with a hetero-atom selected from boron, silicon, phosphorous, sulfur, and/or a halide, or an ion having a −1 or −2 charge and selected from an alkali metal ion and an alkali earth ion.

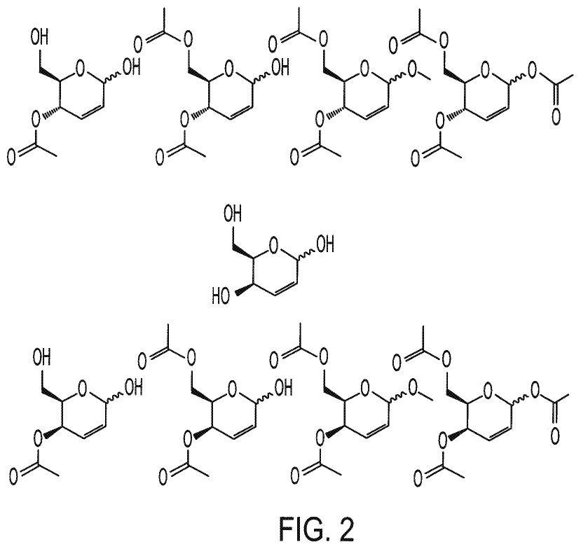

Preferred compositions are shown in FIG. 2 and represented by the following structures:

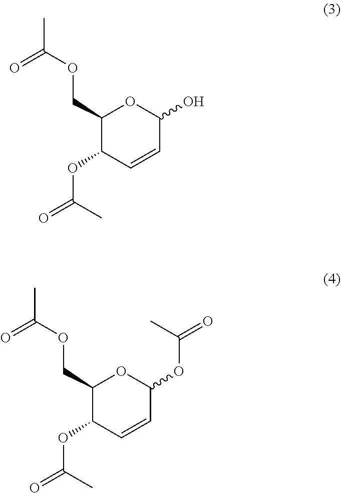 [SMILES: CC(=O)OC[C@H]1OC(O)C=C[C@@H]1OC(C)=O ; CC(=O)OC[C@H]1OC(OC(C)=O)C=C[C@@H]1OC(C)=O]

Such compositions are shown to be effective in treated cancer, or as an adjuvant for treating cancer, especially in human cancer patients.

Another example is a method of treating a subject comprising the following steps:

(a) identifying the subject presenting symptoms of cancer wherein said symptoms warrant treatment with a specific chemotherapeutic agent;

(b) administering therapeutically effective amount the any of the compounds of this disclosure compositions; and

(c) simultaneously, sequentially or separately administering to the subject an effective amount an effective amount of an additional chemotherapeutic agent selected from Table 1.

TABLE 1
Therapeutic Agents

| | | | | | | |
|---|---|---|---|---|---|---|
| Alkyl | MEHD7945 A | AT9283 | XL228 | BI | GSK2636771 | HMPL-013 |
| sufonates | MM-151 | ENMD-2076 | TSU 68, | 847325 | PF-4691502 | KRN951, |
| Ethylenimines | RO5083945, | MK5108 | SU6668 | CI-1040 | PI-103 | AV-951 |
| Nitrogen | GA201 | MLN8237 | ENMD-2076 | GDC-0623 | PKI-587 | MGCD265 |
| mustard | Sym004 | PF-03814735 | AC220 | GDC-0973 | PWT33597 | AEE788, |
| Nitrosoureas | OMP-18R5 | TAK901 | AP24534 | GSK1120 | PX-866 | NVP-AEE788 |
| Platinum | MGAH22 | AS703569, | AS703569, | 212 | SF1126 | AP24534 |
| analogues | MM-111 | R763 | R763 | MEK162 | XL147, | AZD2171 |
| Triazenes | MOAB 2C4 | AT9283 | CEP-701 | PD0325901, | SAR245408 | AZD6474, |
| Folate | PRO132365 | AZD115 | ENMD-0276 | PD-0325901 | XL765, | ZD6474 |
| antagonists | AV 203 | 2-HQPA, | MLN518, | RO5126766 | SAR245409 | BAY 43-9006 |
| Purine | MEHD7945 A | AZD1152 | CT53518 | TAK-733 | BYL719 | BMS-582664 |
| analogues | MM-111 | GSK107 | MP-470 | AMG 208 | GDC-0032 | BMS-690514, |
| Pyrimidine | MM-121, | 0916 | PKC412 | AMG 337 | INK-1117 | EVRI |
| analogues | SAR256212 | PF-03814735 | PLX3397, | ARQ 197 | CAL-101, | E7080 |
| Epothilones | U3-1287, | GSK107 | PLX108-01 | BMS-777607 | GS-1101 | PKC412 |
| Halichondrin | AMG 888 | 0916 | SU-11248 | EMD | LY317615 | RAF265 |
| B analogue | AMG 102 | AMG900 | XL184 | 1214063 | PKC412 | SU-11248 |
| Taxanes | AV-299, | BI | AEE788, | EXEL-2880, | LY317615 | XL184 |
| Vinca | SCH900105 | 847325 | NVP-AEE788 | GSK1363089, | LY317615 | XL820 |
| alkaloids | AMG-479 | PHA-739358 | ARRY 380 | XL880 | LY317615 | YN968D1 |
| Cephalotaxine | BI 836845 | SNS-314 | BIBW-2992 | HMPL-504 | BI 6727 | BAY 57-9352 |
| Camptothecin | BIIB022 | ARQ 736 | CI-1033, | INCB028060, | BI 2536 | E7080 |
| derivatives | CP-751871 | GSK211 | PD183805 | INC280 | GSK461364, | AMG |
| Quinoline | IMC-A12 | 8436 | GSK572016, | MGCD265 | GSK-461364 | 386 |
| alkaloids | MEDI-573 | PLX-4032, | GW2016, | MK-2461 | NMS-1286937 | ABT-263, |
| Anthracenedione | MK-0646 | RG7204, | GW-572016 | MP-470 | ON | ABT-737 |
| Anthracyclines | BI 836845 | R05185426 | HKI-272 | PF-02341066 | 01910.Na | AT101 |
| Epipodophyllo | MEDI-573 | RAF265 | TAK 165 | PF-04217903 | BAY 43-9006 | GDC-0199, |
| toxins | BMS-986015 | RO5212054, | XL647, | XL184 | BAY 73-4506 | ABT-0199, |
| Antitumor | ABT-700 | PLX3603 | KD019 | MGCD265 | LGX818 | RG7601 |
| antibiotic | LY2875358 | AS703569, | BMS-536924 | AP23573, | MLN2480 | GX15-070 |
| Aspariginase | MetMAb, | R763 | BMS-754807 | MK8669 | RO5126766 | G3139 |
| derivatives | R05490258, | AVL-292 | OSI-906 | AY | XL281, | LY2510 |
| Hypomethylating | OA-5D | PCI-32765 | XL228 | 22989, | BMS-908662 | 924 |
| Agents | AMP-224 | HMR-1275, | AZD1480 | SILA | AMG706 | EPZ-5676 |
| Retinoic acid | BMS-936558, | L868275, | CYT-387 | 9268A, | AZD6474, | EPZ-6438, |
| derivatives | MDX-1106, | flavopiridol | INCB018424 | WY-090217 | ZD6474 | E7438 |
| elomerase | ONO-4538 | SCH727 | AT9283 | AZD8055 | BAY 43-9006 | IND |
| template | CT-011 | 965 | BMS-911543 | BEZ235, | BAY 73-4506 | 58359 |
| antagonist | MK-3475 | CYC202 | CEP-701 | NVP-BEZ235 | MP-470 | MK0752 |
| CP675,206 | MDX-1105, | LEE011 | SAR302503, | BGT226, | SU-11248 | FK228 |
| MDX-010, | BMS-936559 | LY2835219 | TG101348 | NVP-BGT226 | XL184 | LBH589 |
| MDX-101, | MPDL3280A, | PD | SB1518 | CCI-779 | PF-02341066 | MK-0683 |
| BMS-734016 | RG7446 | 0332991 | XL019 | GDC-0980 | AB1010 | MS-275, |
| GTX-024 | IMC-3G3 | PLX3397, | SB1518 | MLN0128, | AZD0530 | SNDX-275 |
| MDV3100 | MEDI-575 | PLX108-01 | LY2784544 | INK128 | BMS-354825 | PXD101 |
| CGS 20267 | AMG951, | AEE788, | XL019 | OSI-027 | KX2-391 | OGX-427 |
| FCE-24304 | rhApo2L/ | NVP-AEE788 | CP-690550 | PF-4691502 | XL228 | SAR405 |
| LY-139481 | TRAIL | AP26113 | AB1010 | PI-103 | R788 | 838, MI-773 |
| R-1569 | HGS-ETR1 | AZD6474, | AG-013736 | PKI-587 | MGCD265 | 17-AAG, |
| ACZ885 | AMG-655 | ZD6474 | AMG706 | PWT33597 | XL184 | KOS-953 |
| BMS-986015 | HGS-ETR2 | AZD9291 | AS703569, | RAD001 | LY2157299 | AT13387 |
| AMP-224 | LBY135 | BIBW-2992 | R763 | SF1126 | AZD8055 | AUY922 |
| BMS-936558, | PRO95780 | BPI-2009H | BAY 57-9352 | XL765, | MLN0128, | Debio |
| MDX-1106, | IMC-1121B | CI-1033, | BAY 73-4506 | SAR245409 | INK128 | 0932 |
| ONO-4538 | AMN107 | PD183805 | BMS-354825 | AMG706 | OSI-027 | IPI-504 |
| CT-011 | AP24534 | CO-1686 | GW786034 | BIBF1120 | ABT-869 | STA-9090 |
| MK-3475 | AS703569, | HMPL-813 | MLN518, | GW786034 | ENMD-2076 | XL888 |
| MDX-1105, | R763 | MNPL-309 | CT53518 | MLN518, | EXEL-2880, | MK0752 |
| BMS-936559 | AT9283 | OSI-774, | MP-470 | CT53518 | GSK1363089, | PF-03084014 |
| MPDL3280A, | BMS-354825 | CP-258, 774 | PKC412 | X-82 | XL880 | REGN421 |
| RG7446 | INNO-406, | XL647, | PLX3397, | CP-868569 | HMPL-012 | R04929097, |
| APC8015 | NS-187 | KD019 | PLX108-01 | MP-470 | PTK 787, | R4733 |
| TheraCys | SKI-606 | ZD1839 | STI-571, | PKC412 | CGP | ABT-888 |
| TSF | STI-571, | AZD8931 | CGP57148, | ABT-869 | 79787, | AG-014699, |
| KW-0761 | CGP57148, | BMS-599626, | CGP57148 B | AG-013736 | ZK | PF-01367338 |
| MT103, | CGP57148 | AC480 | SU-11248 | BAY 43-9006 | 222584 | AZD2281 |
| MT-103, | B XL228 | BMS-690514, | XL184 | BAY 57-9352 | TSU 68, | BMN 673 |
| MEDI-538 | AS703569, | EVRI | XL820 | BAY 73-4506 | SU6668 | BSI-201 |
| GA101 | R763 | EKB-569 | INNO-406, | SU-11248 | X-82 | MK4827 |
| IDEC-102, | AZD5363 | HM781-36B | NS-187 | TSU 68, | XL647, | CS 7017, |
| IDEC-C2B8 | GDC-0068 | PF-00299804 | AS703569, | SU6668 | KD019 | RS5444 |
| IDEC Y2B8 | GSK2110183 | XL647, | R763 | XL820 | AG-013736 | LDP 341, |
| CAT-8015 | MK-2206 | KD019 | BVD-523 | BAY 80-6946 | AMG706 | MLN341, |
| SGN-35 | AP26113 | AB1010 | MK-8353, | BEZ235, | BAY 73-4506 | PS-341 |
| Campath | ASP3026 | GSK225 | SCH900353 | NVP-BEZ235 | BIBF1120 | PR-171 |
| IMGN901 | CH5424802, | 6098 | AS703026, | BGT226, | E-3810 | GDC-0449 |
| hLL1 | AF802 | VS-4718 | MSC1936 | NVP-BGT226 | GW786034 | GDC-0449 |
| TRC105 | LDK378 | VS-6063 | 369B | BKM120 | | LDE225 |
| BMS 663513 | PF-02341066 | AB1010 | AZD6244, | BYL719 | | LY2940680 |
| MORAb-004 | TSR-011 | AP24534 | ARRY-142886 | GDC-0941 | | CC-4047 |
| CP675, 206 | X-396 | AZD4547 | AZD8330 | GDC-0980 | | CC-5013 |
| MDX-010, | AS703569, | BGJ398, | BAY 86-9766, | | | |
| MDX-101, | R763 | NVP-BGJ398 | RDEA119 | | | |
| BMS-734016 | | BIBF1120 | | | | |
| MEGF0444A | | E-3810 | | | | |
| ABT-806, | | HMPL-012 | | | | |
| mAb-806 | | TKI258, | | | | |
| ABX-EGF | | CHIR-258 | | | | |
| h-R3, | | | | | | |
| TheraCIM | | | | | | |
| IMC-11F8 | | | | | | |
| IMC-C225 | | | | | | |

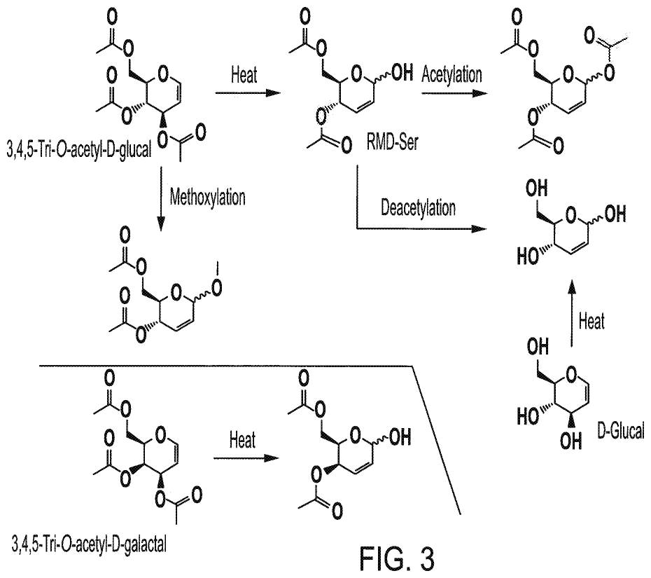

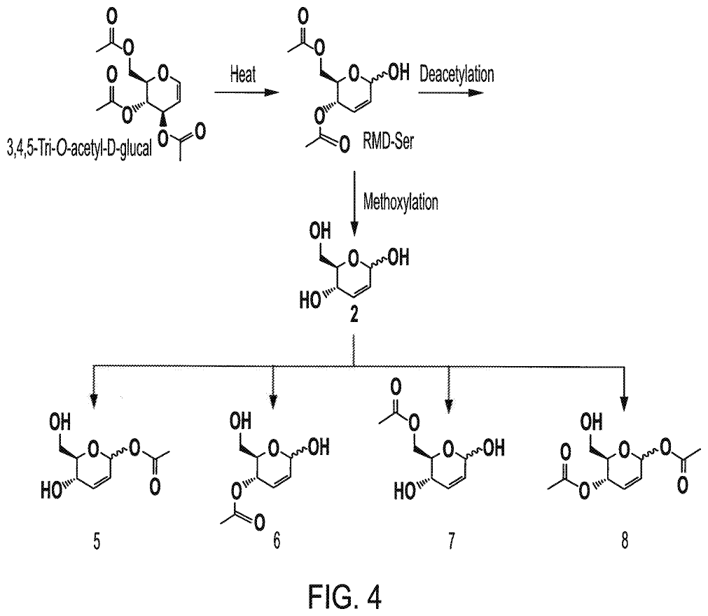

The compounds can be synthesized according to the exemplary reactions depicted in FIGS. 3 and 4. Suitable techniques include subjecting D-glucal, tri-O-aetyl-D-glucal or tri-O-aetyl-D-galactal to elevated temperatures. In preferred examples, 3,4,5-tri-O-aetyl-D-glucal or 3,4,5-tri-O-aetyl-D-galactal are subjected to heat to produce the compounds of this disclosure. Additionally, the compounds can be subjected to deactylation, methoxylation and acetylation according to techniques known in the art to further modify functional groups as desired.

The composition can be administered orally, buccally and/or by oral inhalation. In one instance, the chemotherapeutically active composition can be administered subcutaneously, parenterally, transdermally, intraperitoneally, intramuscularly, by suppository, by implantation, by intravesical instillation, by intraocularly instillation, by intracavitary instillation, by intraarterially instillation, by intralesionally instillation, or by application to non-nasal, non-buccal mucous membranes. In another instance, the chemotherapeutically active composition is administered nasally, by nasal inhalation, by intranasal instillation, by implantation, by intracavitary or intravesical instillation, intraocularly, intraarterially, intralesionally, transdermally, or by application to nasal mucous membranes. In yet another instance, the chemotherapeutically active composition is administered topically.

The cancers that may be treated by the composition of this disclosure include, for example, carcinoma; sarcoma; melanoma; lymphoma; leukemia; brain tumor; cancer found in the blood; cancer found in a tissue in the skin; cancer found in a tissue in the lungs; cancer found in a tissue in the breast; cancer found is the eyes; cancer found in the liver; cancer found in the prostate; or cancer found in a tissue in the pancreas. The method can be used to treat or decrease polyps, total adenoma counts, intestinal tumors, and/or cancer cell proliferation in the subject.

The compounds may be effectively used in the treatment or prevention of conditions associated with cell-specific increases in glucose uptake without a corresponding lactate production. Following treatment with the compounds, lactate production is showed to be increased over the lactate production of the same type of cells prior to treatment.

The compounds may be effectively used in the treatment or prevention of conditions associated with hypoxic cells expressing elevated levels of HIF activity. Following treatment with the compounds of this disclosure, the human has lower HIF activity in the hypoxic cells.

The compounds may be effectively used in the treatment or prevention of conditions associated with a plurality of cells displaying a lower amplitude of circadian oscillation. Following treatment with the compounds of this disclosure, the human has an increase in the amplitude of circadian oscillation in the treated cells.

The compounds may be effectively used in the treatment or prevention of conditions associated with a decrease in reactive oxygen species in mitochondria of the cells of the human. Following treatment with the compounds of this disclosure, the mitochondria of the cells of the human patient are shown to have an increase in reactive oxygen species when compare the levels prior to treatment. The reactive oxygen species is a byproduct of oxidative phosphorylation in the electron transport chain in the mitochondria and the increase in the reactive oxygen species is an effect of increased cellular respiration in the mitochondria.

Additionally, the compounds may be effectively used in the treatment or prevention of Alzheimer's, high cholesterol and diabetes.

Yet another example is a composition that includes any of the above described adjuvants and a pharmaceutically-acceptable carrier. In one instance, the composition further includes an effective amount of one or more chemotherapeutic agents selected from Table 1. In still another instance, the composition further includes a second agent selected from the group consisting of one or more anti-inflammatory agents, antidiabetic agents, hypolipidemic agents, additional chemotherapeutic agents selected from Table 1, antiviral agents, antibiotics, metabolic agents, small molecule inhibitors, protein kinase inhibitors, adjuvants, apoptotic agents, anti-proliferative agents, and organotropic targeting agents, and combinations thereof.

### EXAMPLES

### Synthesis Example 1

3,4,5-Tri-O-aetyl-D-glucal (1.16 g, 4.26 mmol) was dissolved in water (100 mL). The solution was autoclaved at 121 degree for 20 min, and the chilled using an ice-bath. The solvent was lyophilized to remove water completely. The residue was purified by silica gel chromatography (n-hexane/ethyl acetate, 1:1, v/v). Yield 580 mg, 59%. Rf=0.29 and 0.40 (n-hexane/ethyl acetate, 1:1, v/v). The resulting compound is referred to as “RMD-Ser” and shown below as formula (3). RMD-Ser has an equilibrium between dihydropyran form and aldehyde form in solution.

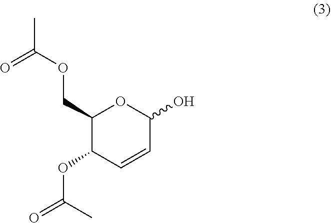 [SMILES: CC(=O)OC[C@H]1OC(O)C=C[C@@H]1OC(C)=O]

### Synthesis Example 2

3,4,5-Tri-O-aetyl-D-galactal (1.16 g, 4.26 mmol) was dissolved in water (100 mL). The solution was autoclaved at 121 degree for 20 min, and the chilled using an ice-bath. The solvent was lyophilized to remove water completely. The residue was purified by silica gel chromatography (n-hexane/ethyl acetate, 1:1, v/v). Yield 580 mg, 59%. Rf=0.29 and 0.40 (n-hexane/ethyl acetate, 1:1, v/v). The resulting compound is shown below.

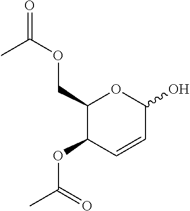 [SMILES: CC(=O)OC[C@H]1OC(O)C=C[C@H]1OC(C)=O]

### In Vitro Example 1

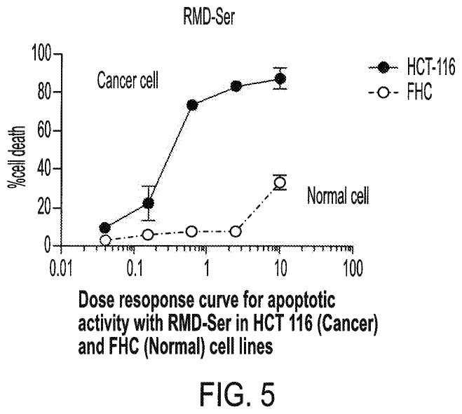

The compounds of Synthesis Example 1 was tested in HCT 116 (cancer) and FHC (normal) cell lines to evaluate apoptotic activity. Dosage was varied and the percent cell death was determined to obtain the dose-response curves as shown in FIG. 5.

Example 1. A composition comprising a molecule represented by formula (1)

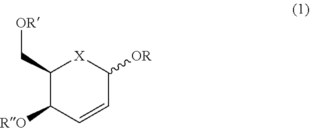

where X is selected from the group consisting of O and S; and where R, R′, and R″ are independently selected from the group consisting of hydrogen, an organic functionality consisting of carbon with hydrogen, nitrogen and/or oxygen and including from 1 to about 24 carbon atoms, a hetero-organic functionality comprising carbon with hydrogen, nitrogen and/or oxygen, with a hetero-atom selected from boron, silicon, phosphorous, sulfur, and/or a halide, or an ion having a −1 or −2 charge and selected from an alkali metal ion and an alkali earth ion.

Example 2. A composition wherein a substantial portion of composition is comprised of molecules represented by formula (2)

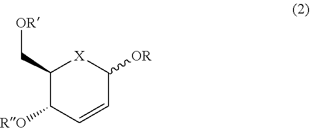

where X is selected from the group consisting of O and S; and where R, R′, and R″ are selected from the group consisting of hydrogen, an organic functionality consisting of carbon with hydrogen, nitrogen and/or oxygen and including from 1 to about 24 carbon atoms, a hetero-organic functionality comprising carbon with hydrogen, nitrogen and/or oxygen, with a hetero-atom selected from boron, silicon, phosphorous, sulfur, and/or a halide, or an ion having a −1 or −2 charge and selected from an alkali metal ion and an alkali earth ion; but when X is oxygen and R and R′ are hydrogen: R″ is selected from the group consisting of an organic functionality, hetero-organic functionality, or an ion having a −1 or −2 charge, and when R′ and R″ are acetyl groups (—COCH3): R is not an ethyl group.

Example 3. The composition of example 2, wherein R is a hydrogen atom.

Example 4. The composition of any one of the preceding examples, wherein R′ is an acetyl group.

Example 5. The composition of any one of the preceding examples, wherein R″ is an acetyl group.

Example 6. The composition of any one of the preceding examples, wherein R′ includes a carbonyl group.

Example 7. The composition of any one of the preceding examples, wherein R″ includes a carbonyl group.

Example 8. The composition of example 2 where the molecules represented by formula (2), are represented by formula (5):

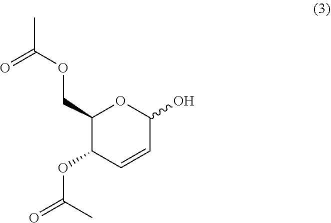 [SMILES: CC(=O)OC[C@H]1OC(O)C=C[C@@H]1OC(C)=O]

Example 9. The composition of example 2 where the molecules represented by formula (2), are also represented by the formula (4):

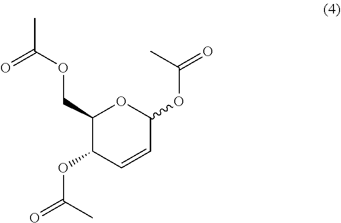 [SMILES: CC(=O)OC[C@H]1OC(OC(C)=O)C=C[C@@H]1OC(C)=O]

Example 10. A composition comprising a molecule represented by either one of formulas (5) and (6):

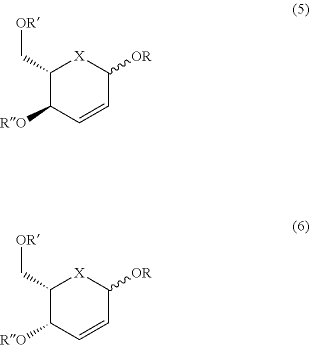

where X is selected from the group consisting of O and S; and where R, R′, and R″ in each of formula (1) and (2) are independently selected from the group consisting of hydrogen, an organic functionality consisting of carbon with hydrogen, nitrogen and/or oxygen and including from 1 to about 24 carbon atoms, a hetero-organic functionality comprising carbon with hydrogen, nitrogen and/or oxygen, with a hetero-atom selected from boron, silicon, phosphorous, sulfur, and/or a halide, and an ion having a −1 or −2 charge and selected from an alkali metal ion and an alkali earth ion.

Example 11. A pharmaceutical composition comprising: a compound represented by any one of formulas (1), (2), (5), or (6):

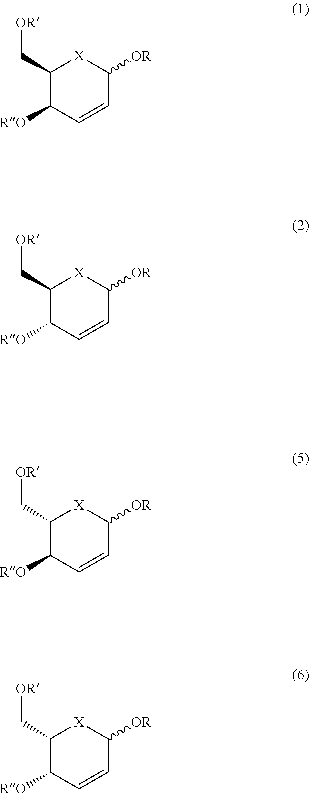

where X in each formula (1), (2), (5), and (6) is independently selected from the group consisting of O and S; where R, R′, and R″ in each of formula (1), (2), (5), and (6) are independently selected from the group consisting of hydrogen, an organic functionality consisting of carbon with hydrogen, nitrogen and/or oxygen and including from 1 to about 24 carbon atoms, a hetero-organic functionality comprising carbon with hydrogen, nitrogen and/or oxygen, with a hetero-atom selected from boron, silicon, phosphorous, sulfur, and/or a halide, and an ion having a −1 or −2 charge and selected from an alkali metal ion and an alkali earth ion; and also comprising at least one of a pharmaceutically acceptable adjuvant, a pharmaceutically acceptable binder, and a pharmaceutically acceptable carrier.

Example 12. The pharmaceutical composition of example 11, further comprising an effective amount at least one chemotherapeutic agent.

Example 13. The pharmaceutical composition of example 12, wherein the chemotherapeutic agent is listed on Table 1.

Example 14. The pharmaceutical composition of example 11, wherein the pharmaceutical composition is orally active.

Example 15. The pharmaceutical composition of example 11, wherein the pharmaceutical composition is formulated in a tablet, capsule, or oral inhaler.

Example 16. A process of treating a patient wherein the patient is administered a pharmaceutical composition of any one of examples 11-15.

Example 17. The process of example 16, wherein the patient is human.

Example 18. The process of example 16, wherein the human is displaying a cell-specific increases in glucose uptake without a corresponding lactate production.

Example 19. The process of example 18, wherein the following treatment lactate production is showed to be increased over the lactate production of the same type of cells prior to treatment.

Example 20. The process of example 16, wherein the human has hypoxic cells expressing elevated levels of HIF activity.

Example 21. The process of example 20, wherein following treatment the human has lower HIF activity in the hypoxic cells.

Example 22. The process of example 16, wherein the human possesses a plurality of cells displaying a lower amplitude of circadian oscillation.

Example 23. The process of example 22 wherein the following treatment the human has an increasing the amplitude of circadian oscillation in the treated cells.

Example 24. The process of example 16, wherein the human has a decrease in reactive oxygen species in mitochondria of the cells of the human.

Example 25. The process of example 24, wherein following treatment the mitochondria of the cells of the human patient are shown to have an increase in reactive oxygen species when compared to the levels prior to treatment; wherein the reactive oxygen species is a byproduct of oxidative phosphorylation in the electron transport chain in the mitochondria; wherein the increase in the reactive oxygen species is an effect of increased cellular respiration in the mitochondria.

Example 26. A method of treating a subject comprising the following steps: (a) identifying the subject presenting symptoms of cancer wherein said symptoms warrant treatment with a specific chemotherapeutic agent; (b) administering an effective amount the composition of any of examples 1-10; (c) simultaneously, sequentially or separately administering to the subject an effective amount an effective amount of the chemotherapeutic agent of step (a).

Example 27. The method of example 26, wherein during the identification step (a) it is determined that at least one of the chemotherapeutic agents in Table 1 would be beneficial and such agent is administered in step (c).

Example 28. The method of example 26, wherein the subject's symptoms of cancer have been shown to more effectively reduced by treatment with the chemotherapeutic agent of step (c) and the adjuvant than by the chemotherapeutic agent of step (c) alone in a controlled study comparing the effectiveness of the chemotherapeutic agent of step (c) and the effectiveness of chemotherapeutic agent of step (c) and the adjuvant.

Example 29. The method of example 26, wherein the adjuvant is administered orally, buccally and/or by oral inhalation.

Example 30. The method of example 26, wherein the adjuvant is administered subcutaneously, parenterally, transdermally, intraperitoneally, intramuscularly, by suppository, by implantation, by intravesical instillation, by intraocularly instillation, by intracavitary instillation, by intraarterially instillation, by intralesionally instillation, or by application to non-nasal, non-buccal mucous membranes.

Example 31. The method of example 26, wherein the adjuvant is administered nasally, by nasal inhalation, by intranasal instillation, by implantation, by intracavitary or intravesical instillation, intraocularly, intraarterially, intralesionally, transdermally, or by application to nasal mucous membranes.

Example 32. The method of example 26, wherein the adjuvant is administered topically.

Example 33. The method of example 26, wherein the cancer of step (a) is further diagnosed as carcinoma.

Example 34. The method of example 26, wherein the cancer of step (a) is further diagnosed sarcoma.

Example 35. The method of example 26, wherein the cancer of step (a) is further diagnosed melanoma.

Example 36. The method of example 26, wherein the cancer of step (a) is further diagnosed lymphoma.

Example 37. The method of example 26, wherein the cancer of step (a) is further diagnosed leukemia.

Example 38. The method of example 26, wherein the cancer of step (a) is found in the blood.

Example 39. The method of example 26, wherein the cancer of step (a) is found in a tissue in the skin.

Example 40. The method of example 26, wherein the cancer of step (a) is found in a tissue in the lungs.

Example 41. The method of example 26, wherein the cancer of step (a) is found in a tissue in the breast.

Example 42. The method of example 26, wherein the cancer of step (a) is found in a tissue in the pancreas.

Example 43. The method of example 26, wherein the treating decreases polyps, total adenoma counts, intestinal tumors, and/or cancer cell proliferation in the subject.

Example 44. The method of example 26, wherein the treating increases cancer cell apoptosis.

Example 45. The method of example 26, wherein the subject is a human or mammal subject.

Example 46. The pharmaceutical composition of example 11 further comprising a second agent selected from the group consisting of one or more anti-inflammatory agents, antidiabetic agents, hypolipidemic agents, additional chemotherapeutic agents selected from Table 1, antiviral agents, antibiotics, metabolic agents, small molecule inhibitors, protein kinase inhibitors, adjuvants, apoptotic agents, anti-proliferative agents, and organotropic targeting agents, and combinations thereof.

Herein, the compositions represented by formulas (1), (2), (5), and (6):

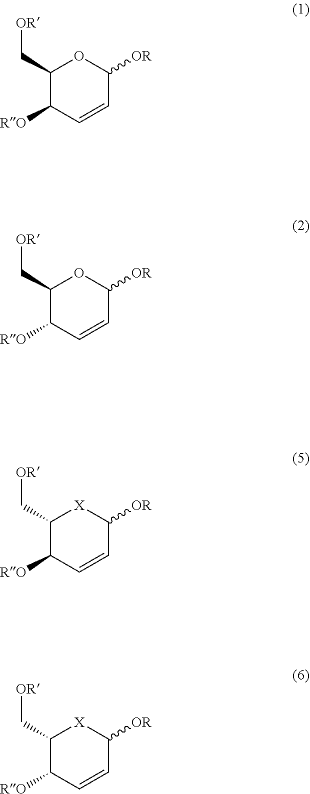

wherein R is a hydrogen atom (H), can further be represented by the “ring-opened” tautomers (1′), (2′), (5′), and (6′):

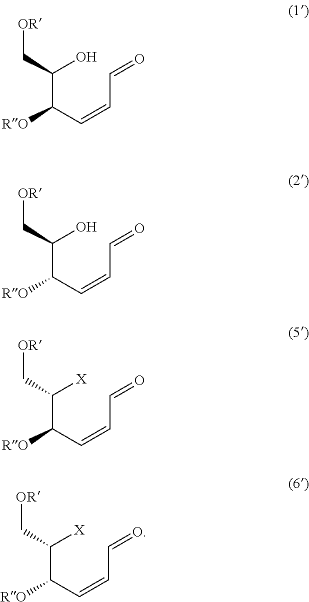

In such tautomers, X can be a hydroxyl (—OH) or a thiol (—SH) functionality, preferably, X is a hydroxyl.

1. A composition comprising a compound of formula (1) or a ring-opened tautomer thereof: 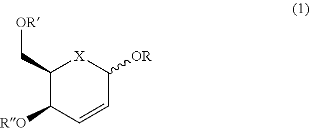
where X is selected from the group consisting of O and S; and
where R, R′, and R″ are independently selected from the group consisting of hydrogen,
an organic functionality consisting of carbon with hydrogen, nitrogen and/or oxygen and including from 1 to about 24 carbon atoms,
a hetero-organic functionality comprising carbon with hydrogen, nitrogen and/or oxygen, with a hetero-atom selected from boron, silicon, phosphorous, sulfur, and/or a halide, or
an ion having a −1 or −2 charge and selected from an alkali metal ion and an alkali earth ion; and
wherein when the compound of formula (1) is a ring-opened tautomer then R is hydrogen.

2. A composition wherein a substantial portion there of comprises compounds represented by formula (2) or ring-opened tautomers thereof: 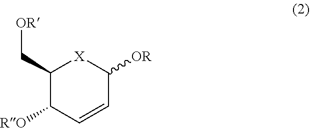
where X is selected from the group consisting of O and S; and
where R, R′, and R″ are selected from the group consisting of
hydrogen,
an organic functionality consisting of carbon with hydrogen, nitrogen and/or oxygen and including from 1 to about 24 carbon atoms,
a hetero-organic functionality comprising carbon with hydrogen, nitrogen and/or oxygen, with a hetero-atom selected from boron, silicon, phosphorous, sulfur, and/or a halide, or
an ion having a −1 or −2 charge and selected from an alkali metal ion and an alkali earth ion;
but
when X is oxygen and R and R′ are hydrogen: R″ is selected from the group consisting of an organic functionality, hetero-organic functionality, or an ion having a −1 or −2 charge, and
when R′ and R″ are acetyl groups (—COCH₃): R is not an ethyl group; and
wherein when the compound of formula (2) is a ring-opened tautomer then R is hydrogen.

3. The composition of claim 2, wherein R is a hydrogen atom.

4. The composition of claim 2, wherein R′ is an acetyl group.

5. The composition of claim 2, wherein R″ is an acetyl group.

6.-7. (canceled)

8. The composition of claim 2 where the compounds represented by formula (2), are represented by formula (3) or a ring-opened tautomer thereof:

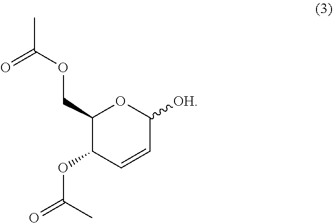 [SMILES: CC(=O)OC[C@H]1OC(O)C=C[C@@H]1OC(C)=O]

9.-10. (canceled)

11. A pharmaceutical composition comprising:
a compound represented by any one of formulas (1), (2), (5), or (6) or a ring-opened tautomer thereof: 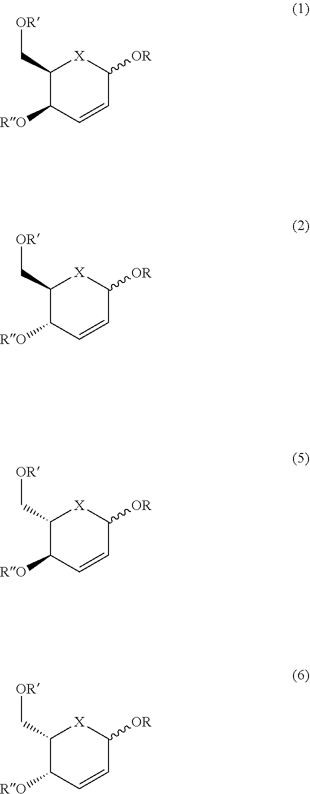
where X in each formula (1), (2), (5), and (6) is independently selected from the group consisting of O and S;
where R, R′, and R″ in each of formula (1), (2), (5), and (6) are independently selected from the group consisting of hydrogen, an organic functionality consisting of carbon with hydrogen, nitrogen and/or oxygen and including from 1 to about 24 carbon atoms, a hetero-organic functionality comprising carbon with hydrogen, nitrogen and/or oxygen, with a hetero-atom selected from boron, silicon, phosphorous, sulfur, and/or a halide, and an ion having a −1 or −2 charge and selected from an alkali metal ion and an alkali earth ion; and
also comprising at least one of a pharmaceutically acceptable adjuvant, a pharmaceutically acceptable binder, and a pharmaceutically acceptable carrier; and
wherein when the compound is a ring-opened tautomer then R is hydrogen.

12. The pharmaceutical composition of claim 11, further comprising a therapeutically effective amount at least one chemotherapeutic agent.

13.-15. (canceled)

16. A process of treating a patient comprising administering a therapeutically effective amount of the pharmaceutical composition of claim 11, wherein the patient is a mammal.

17. (canceled)

18. The process of claim 16, wherein the human is displaying a cell-specific increase in glucose uptake without a corresponding lactate production.

19. (canceled)

20. The process of claim 16, wherein the human has hypoxic cells expressing elevated levels of HIF activity.

21. (canceled)

22. The process of claim 16, wherein the human possesses a plurality of cells displaying a lower amplitude of circadian oscillation.

23. (canceled)

24. The process of claim 16, wherein the human has a decrease in reactive oxygen species in mitochondria of the cells of the human.

25.-46. (canceled)

47. The composition of claim 1, where the compounds represented by formula (1), are represented by the formula, or a ring-opened tautomer thereof:

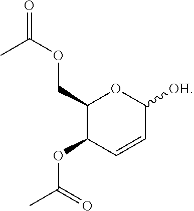 [SMILES: CC(=O)OC[C@H]1OC(O)C=C[C@H]1OC(C)=O]

48. A method of treating a cancer in a subject including the step of administering a compound represented by any one of formulas (1), (2), (5), or (6), or a ring-opened tautomer thereof: 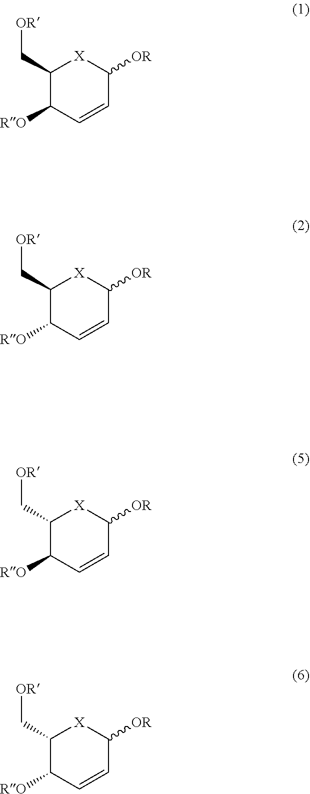
where X in each formula (1), (2), (5), and (6) is independently selected from the group consisting of O and S;
where R, R′, and R″ in each of formula (1), (2), (5), and (6) are independently selected from the group consisting of hydrogen, an organic functionality consisting of carbon with hydrogen, nitrogen and/or oxygen and including from 1 to about 24 carbon atoms, a hetero-organic functionality comprising carbon with hydrogen, nitrogen and/or oxygen, with a hetero-atom selected from boron, silicon, phosphorous, sulfur, and/or a halide, and an ion having a −1 or −2 charge and selected from an alkali metal ion and an alkali earth ion; and
wherein when the compound is a ring-opened tautomer then R is hydrogen.

49. The method of claim 48, wherein the administration increases cancer cell apoptosis.
